(19) United States
(12) Patent Application Publication
(10) Pub. No.: US 2007/0271117 A1
(43) Pub. Date: Nov. 22, 2007

(54) Electronic patient care system

(76) Inventors: Matias A. Klein, Southampton, PA (US); Julian Klein, Huntingdon Valley, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/438,380

(22) Filed: May 22, 2006

(51) Int. Cl. G06Q 10/00; A61B 5/00

(52) U.S. Cl. 705/2; 600/300

(57) ABSTRACT

A patient care system includes a clinician for providing the health care to the patient, an input device for signaling that the patient is initiating a current health care encounter to provide a patient-initiated encounter signal, and current patient information provided by the patient and received by the patient care system in response to the patient-initiated encounter signal. The patient care system includes an interface device in communication with the input device for providing a clinician-notification signal in response to the patient-initiated encounter signal to permit the clinician to determine that the patient has initiated the encounter, a medical records system in communication with the input device for receiving the current patient information from the patient and input devices in communication with medical records system for receiving the current patient information from a plurality of differing patients. The differing patients are disposed at a plurality of differing locations.

### BACKGROUND OF THE INVENTION

1. Field of Invention

This invention relates to the field of health care and, in particular, to the field of providing health care to a patient using telecommunications equipment.

2. Description of Related Art

All references cited herein are incorporated herein by reference in their entireties.

U.S. Pat. Nos. 5,924,074 and 6,347,329 both entitled, “Electronic Medical Records System” issued to Evans on Jul. 13, 1999 and Feb. 12, 2002, respectively, teach a medical record system that created and maintained patient data. The Evans system captured patient data, such as patient complaints, lab orders, medications, diagnoses and procedures using a graphical user interface. Authorized health care providers could access and alter the captured patient data using the system taught by Evans. However, in the Evans system a physician actuated access to the records at the point of care rather than the patient.

Thus, within the Evans system, patients were required to make appointments, sometimes for long periods of time in the future. Furthermore, when the patients arrived at the physician's office for their medical care they may have experienced long waiting periods due to factors such as failures in maintaining the schedules of the physician's offices and over scheduling of patients by the physician's office. Furthermore, prior art such as Evans required employees at the physician's office to pull and refile charts, to perform the scheduling, and many other functions. Under these circumstances doctor's offices may have required two to five full-time employees per physician. Also, these methods often required patients to make trips to the drug store after the visit to the doctor's office if prescriptions were required, thereby resulting in more lost time and inconvenience for the patients.

U.S. Pat. No. 5,416,695 entitled “Method and Apparatus for Alerting Patients and Medical Personnel of Emergency Medical Situations” issued to Stutman et al. on May 16, 1995 teaches a system that enabled a doctor to remotely set selections and limit parameters pertaining to specific medical and geodetic information of a patient. The doctor could also receive updates of the information over a wireless communication network when the parameters have been met. The host computer could extract selected portions of the information in response to the parameters provided over the communication network. A user interface in the Stutman system provided a series of menus on a display. The user interface also provided for manipulation by a keyboard to allow a user to enter the selection and limit parameters in a user recognizable format.

U.S. Pat. No. 3,872,448 entitled “Hospital Data Processing System” issued to Mitchell on Mar. 18, 1975 teaches a system for processing hospital data, such as patient information and biological test information. A central data processing apparatus stored and processed the data and provided for storage of new patient information. A plurality of remote stations could be provided to permit data collection and inputting into the central storage area in the Mitchell system. Memory was provided in the Mitchell system for short term storage of daily transaction data.

U.S. Pat. No. 4,315,309 entitled “Integrated Medical Test Data Storage and Retrieval System” issued to Coli on Feb. 9, 1982 teaches a patient report generating system for receiving, storing and reporting medical test data for a patient population. The system taught by Coli could periodically provide individual patient reports containing test results for each patient. The reports could contain all of the test results for a patient throughout the entire time period during which the patient was a member of the patient population.

U.S. Pat. No. 5,065,315 entitled “System and Method for Scheduling and Reporting Patient Related Services Including Prioritizing Services” issued to Garcia on Nov. 12, 1991 teaches a computerized hospital system for entering information which was pertinent to a patient in the hospital. Initial information was entered as part of the admitting procedure. The initial information included the patient history and the results of the examination ordered by the admitting physician. Additionally, the initial information that was entered into the Garcia system could include orders from a physician for tests or hospital services to be performed. The Garcia system could also schedule hospital services for the patient including the scheduling of tests, x-rays, ultrasound etc. Notes written by the physicians and nurses and their findings and observations were entered into the Garcia system.

U.S. Pat. No. 5,099,424 entitled “Model User Application System for Clinical Data Processing that Tracks and Monitors a Simulated Out-Patient Medical Practice Using Database Management Software” issued to Schneiderman on Mar. 24, 1992 teaches a method for entering new data representative of physician requests, for example requests for blood work or x-rays. Separate data fields were provided in the Schneiderman system for holding data that identified the types of specialists ordering the tests and the general recommendations of the specialists. A report generation program combined salient clinical data from office visits with the test results ordered during the visits. A further report generation program within the Schneiderman system combined salient clinical data from office visits with the results from specialist visits to which the primary care physician referred the patients.

U.S. Pat. No. 5,277,188 entitled “Clinical Information Reporting System” issued to Selker on Jan. 11, 1994 teaches a clinical information reporting system for use with an electronic data base in a health care facility. The electronic data base in the Selker system contained records for a group of patients and operational information. The patient records and operational information were accessed by clinicians and used during the course of the operations of the facility. The patient records included clinical information for a group of patients. Furthermore, a method for generating reports from the information stored in the data base on the care given to the patrons was provided in the Selker system.

U.S. Pat. No. 5,659,741 entitled “Computer System and Method for Storing Medical Histories Using a Carrying Size Card” issued to Eberhardt on Aug. 19, 1997 teaches a system for storing individual medical histories on a storage device. Preferably the size of the storage device was approximately the size of a credit card. New medical data could be added to the storage device as required. Furthermore, information in the storage device could be communicated to other computers in order to permit the information to be available to the health care providers. This permitted retrieval of large data records about the individual by the provider. Additionally, Eberhardt discloses that a second computer can collate and sort data relating to an individual.

### BRIEF SUMMARY OF THE INVENTION

A patient care system for providing health care to a patient includes a clinician for providing the health care to the patient, an input device for signaling by the patient that the patient is initiating a current health care encounter to provide a patient-initiated encounter signal, and current patient information provided by the patient and received by the patient care system in response to the patient-initiated encounter signal. The patient care system includes an interface device in communication with the input device for providing a clinician-notification signal in response to the patient-initiated encounter signal to permit the clinician to determine that the patient has initiated the current health care encounter, an electronic medical records system in communication with the input device for receiving the current patient information from the patient and a plurality of input devices in communication with the electronic medical records system for receiving the current patient information from a plurality of differing patients.

The differing patients of the plurality of differing patients are disposed at a plurality of differing locations. Corporate headquarters receives the current patient information and manages the patient care system in accordance with the current patient information and previous patient information stored in the electronic medical records system. Information is received by the corporate headquarters from the electronic medical records system for managing the patient care system in accordance with the previous patient information and the corporate headquarters manages the patient care system in accordance with both the current patient information and the previous patient information.

The electronic medical records system is in communication with the input device by way of an internet. The electronic medical records system is in communication with the clinician by way of the internet, and current encounter information is received by the clinician from the electronic medical records system in response to the patient-initiated encounter signal. Previous encounter information is provided in accordance with a previous encounter wherein the clinician receives the previous encounter information from the electronic medical records system in response to the patient-initiated encounter signal. Current encounter information is received by the clinician from the electronic medical records system in response to the patient-initiated encounter signal.

A kiosk houses the input device, and the kiosk is a permanent kiosk, a temporary kiosk or a pop up kiosk. The current patient information is patient identification information, patient symptom information and patient insurance information.

### BRIEF DESCRIPTION OF SEVERAL VIEWS OF THE DRAWINGS

The invention will be described in conjunction with the following drawings in which like reference numerals designate like elements and wherein:

FIG. 1 shows a schematic representation of the electronic patient care system and method of the present invention.

FIGS. 2A-J show images including schematic representations of sample screen shot images illustrating some of the steps that can be performed in accordance with one of the preferred embodiment of the present invention, for example, as set forth in FIG. 1.

FIG. 3 shows a screen shot representation of an example of the type of data that can be displayed to a clinician in the system and method of the present invention, for example, as set forth in FIG. 1.

FIG. 4 shows a screen shot representation of a prescription form which can be used by a clinician to write prescriptions for a patient in accordance with the system of the present invention, for example, as set forth in FIG. 1.

FIG. 5 is a screen shot representation of further information, for example, queue information, that can be displayed to a clinician in the system of the present invention, for example, as set forth in FIG. 1.

FIG. 6 shows a screen shot representation of further information that can be stored within the system of the present invention and displayed to a clinician, for example, as set forth in FIG. 1.

FIG. 7 shows a screen shot representation of a lab test report for a patient that can be stored within the system of the present invention and displayed to a clinician, for example, as set forth in FIG. 1.

### DETAILED DESCRIPTION OF THE INVENTION

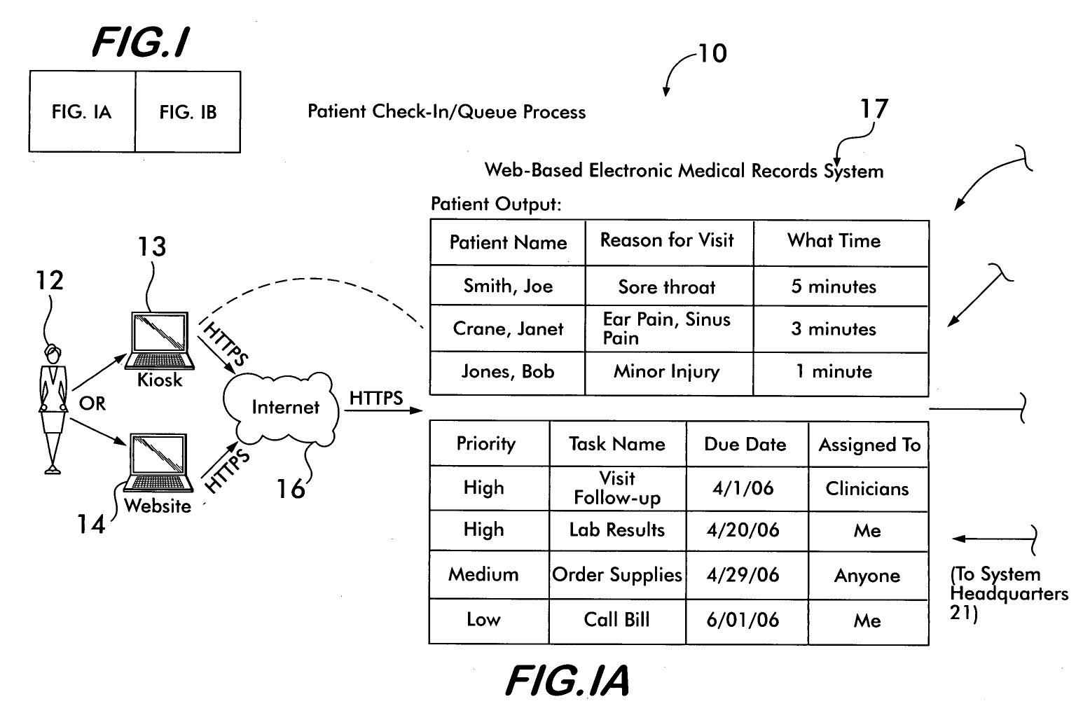

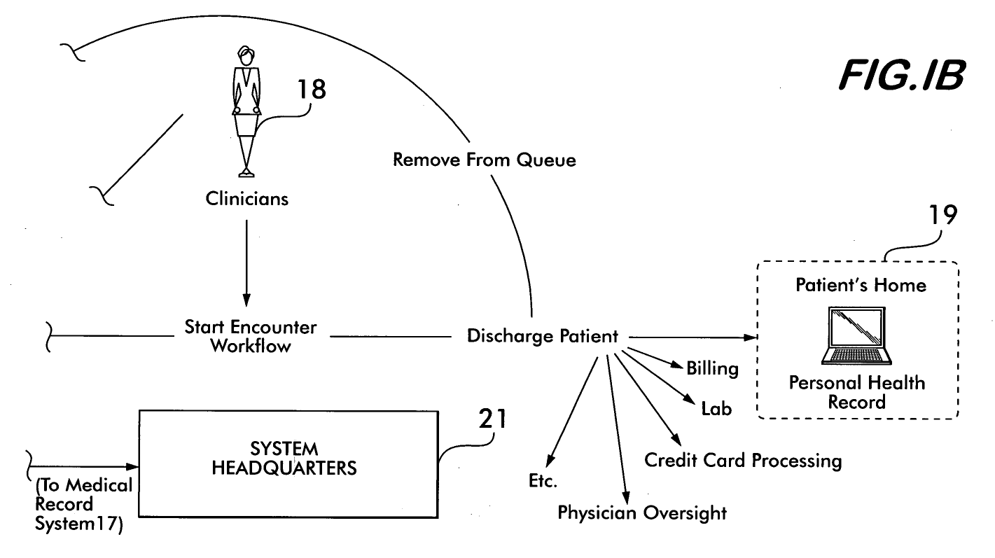

Referring now to FIG. 1, there is shown a schematic representation of the electronic patient care system 10 of the present invention. A patient 12 can register with the electronic patient care system 10 to initiate an encounter by accessing a web-based electronic medical records system 17. The electronic medical records system 17 can be a centralized or a distributed data center and can contain all of the information of the electronic patient care system 10. This permits encounters between the patient 12 and a clinician 18 for permitting the patient 12 to receive health care services from the clinician 18, wherein the clinician 18 is also in direct communication with the electronic medical records system 17.

The accessing of the electronic medical records system 17 by the patient 12 can be by way of the internet 16 using a kiosk 13 or any other type of information device or information interface device, a website 14 or any other type of internet interface known to those skilled in the art. The patient care system 10 can thus permit direct and immediate transmission of information between the patient 12, the clinician 18 and the web-based electronic medical records system 17. Thus, within the electronic patient care system 10 the kiosks 13 are fully integrated with the electronic medical records system 17.

The kiosk 13 can be part of a kiosk system. A kiosk system can include all of the facilities and equipment necessary to permit the patient 12 to initiate an encounter and receive health care from the clinician 18 within the patient care system 10, as discussed in more detail below. For example, a kiosk system can include two or more examination rooms, in addition to two or more kiosks 13 each having its own display device 15. Preferably each clinician 18 can be provided with a separate examination room for working with individual patients 12 in privacy. In a preferred embodiment of the invention, a kiosk system suitable for two clinicians 18 can be approximately two hundred square feet. However, the amount of space per clinician 18 can vary as needed. Accordingly, a kiosk system according to the present invention can be located in a supermarket near supermarket pharmacy, in a strip mall or in any other convenient location. Likewise, it will be understood by those skilled in the art than the website 14 can be part of a website system having all of the features described herein with respect to the kiosk system for providing an encounter between the patient 12 and the clinician 18.

It will be understood by those skilled in the art that any number of kiosk systems can be disposed at different locations while they are coupled to the medical records system 17 in order to practice the invention at each of the kiosk systems. For example, the kiosk systems can be located in different cities within a single country or even in different countries. Additionally, the patients 12 can be coupled to the medical records system 17 by way of any other transmission channel known to those skilled in the art. For example, the patients 12 can be coupled to the medical records system 17 by way of CDMA communications systems. For example, web technology enabled cell phones, such as smart phones, can permit the patients 12 to indicate their intention to initiate an encounter with the clinician 18 and send registration information to the clinician 18. An interactive television with an internet enabled set top box can also be used. An IVR phone system to power the same process.

All information within the patient care system 10 can be displayed for real-time viewing by management. The analysis is mostly for business intelligence applications located within the corporate headquarters 21. Additionally, at the corporate headquarters 21 observation of data flow and analytics can be performed on the information within the medical records system 17 by the management of the patient care system 10. Furthermore, the analytics can be performed in real time based upon the information received at the corporate headquarters 21 and, accordingly, the patient care system 10 can be managed in real-time from within the corporate headquarters 21. Inventory can be tracked, contracts can be entered with suppliers and pricing decisions can be made. The results of the real-time analytics can be shared with drug companies or insurance companies or any other legitimate corporations.

Thus, managers located at the corporate headquarters 21 can do analytics on the data by logging into the system website from their corporate headquarters. However, it will be understood that any real-time analysis performed in this manner need not be part of the patient care process at the time of the encounter. Rather, the analysis performed can be performed at any time, primarily for the purpose of business intelligence applications.

For example, if a plurality of kiosk systems, which can have one or more kiosks 13, are disposed at different locations they can all be coupled to the medical records system 17. The lengths of the queues at each of the various kiosks 13 can be determined by algorithms preferably located in the data center 17 of the system 10. Accordingly, the patients 12 waiting in longer queues at some of the kiosks 13 can be informed of any shorter queues at other kiosks 13 that are located nearby by the system 10. The determinations of which kiosks 13 have longer queues, which kiosks 13 are near the kiosks 13 which have long queues, whether the nearby queues are shorter than the ones having long queues and the likely travel time from a longer queue to a shorter queue can all be performed at any convenient location.

The medical records system 17 may store any type of information on the patient 12 that was previously received and recorded, as well as any new information provided by the patient 12 to the medical records system 17 during a current encounter with the patient care system 10. For example, the patient 12 can transmit current information on his/her complaints and symptoms to the medical records system 17 by way of the kiosk 13. The previously provided information and the currently provided information within the medical records system 17 can be displayed on a graphical user interface which is available for viewing by the clinician 18 at the location of the patient 12. The choice of information to be displayed in this manner can be interactively controlled by the clinician 18 using the graphical user interface at the location of the patient 12.

Using the current and previous information stored within the electronic medical records system 17, as well as any verbal reports provided by the patient 12 to the clinician 18, the clinician 18 can provide health care to the patient 12 during a current encounter. The health care is provided during the current encounter according to the new and previous information of the patient 12 and the medical training of the clinician 18. The information that the patient 12 can provide in order to initiate an encounter with the patient care system 10 are described in more detail below. It will be understood that the clinician 18 can initiate an encounter with the health care system 10.

After completing an encounter within the patient care system 10 a patient 12 can be discharged by the clinician 18. Information and instructions based upon the encounter can be sent by the clinician 18 to the personal health records of the patient 12 at the home of the patient 12, for example by email. In another embodiment educational information for the patient 12 related to encounters with the health care system 10 can be stored in the same server as the electronic medical records system 17 and a notification that new information is in the personal health record of the patient 12 can be sent via email to the patient 12. Information with respect to an encounter can also be sent to the billing offices to arrange for billing, to laboratories in order to request lab work, and to collaborating physicians who can provide oversight of the encounters within the patient care system 10. Additionally, information with respect to an encounter can be sent to any other location or stored for on-demand retrieval. Retrieval can be from any location where the information within the medical records system 17 on previous or current encounters between the system 10 and the patient 12 may be useful.

Thus, it will be understood that the kiosk 13, the medical records system 17, the billing offices, the pharmacy, the laboratories and the collaborating physicians are in direct, real time communication with each other and are, therefore, fully integrated with each other. In a preferred embodiment pharmacies and laboratories can be integrated with the patient care system 10 by connecting them to the system 10 via a clearing-house or other conduit rather than being a part of the system 10. Furthermore, it will be understood that any other location that may be useful for facilitating the performance of the operations of the patient care system 10 can also be integrated with the patient care system 10 or connected them to the system 10 via the clearing-house or other conduit, provided it can be placed in with the system 10 as understood by those skilled in the art.

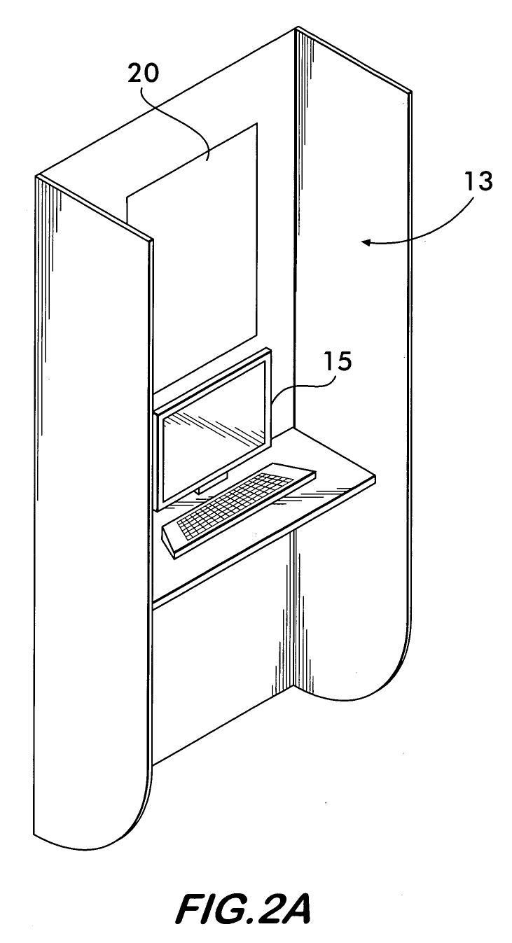

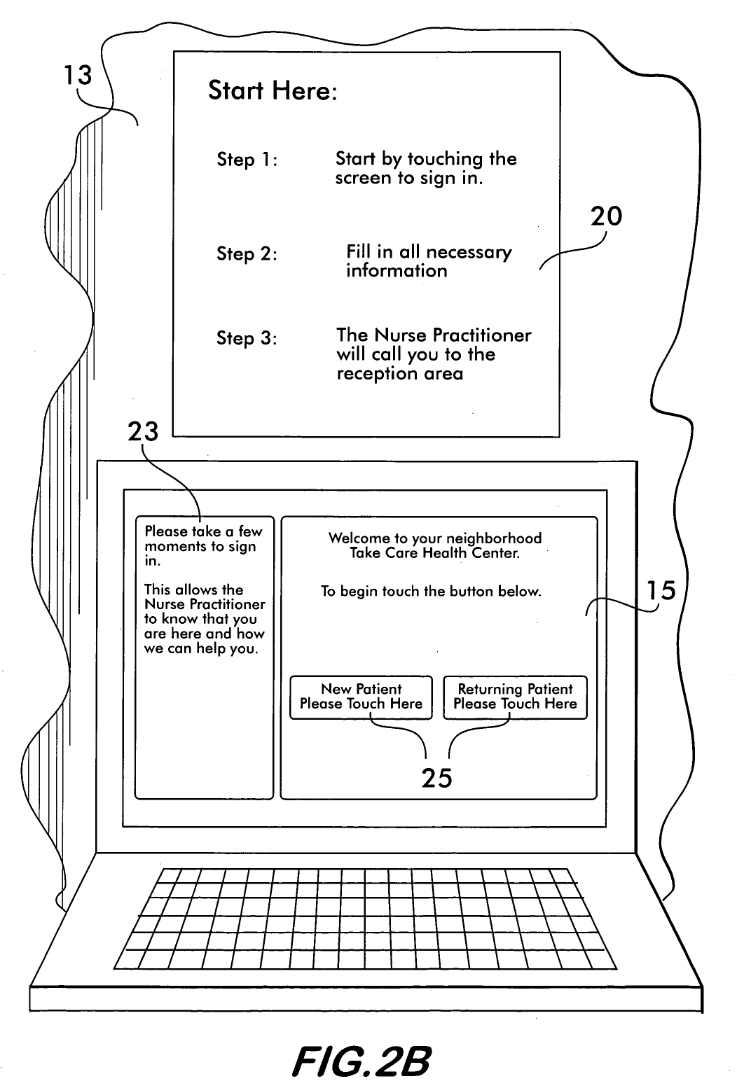

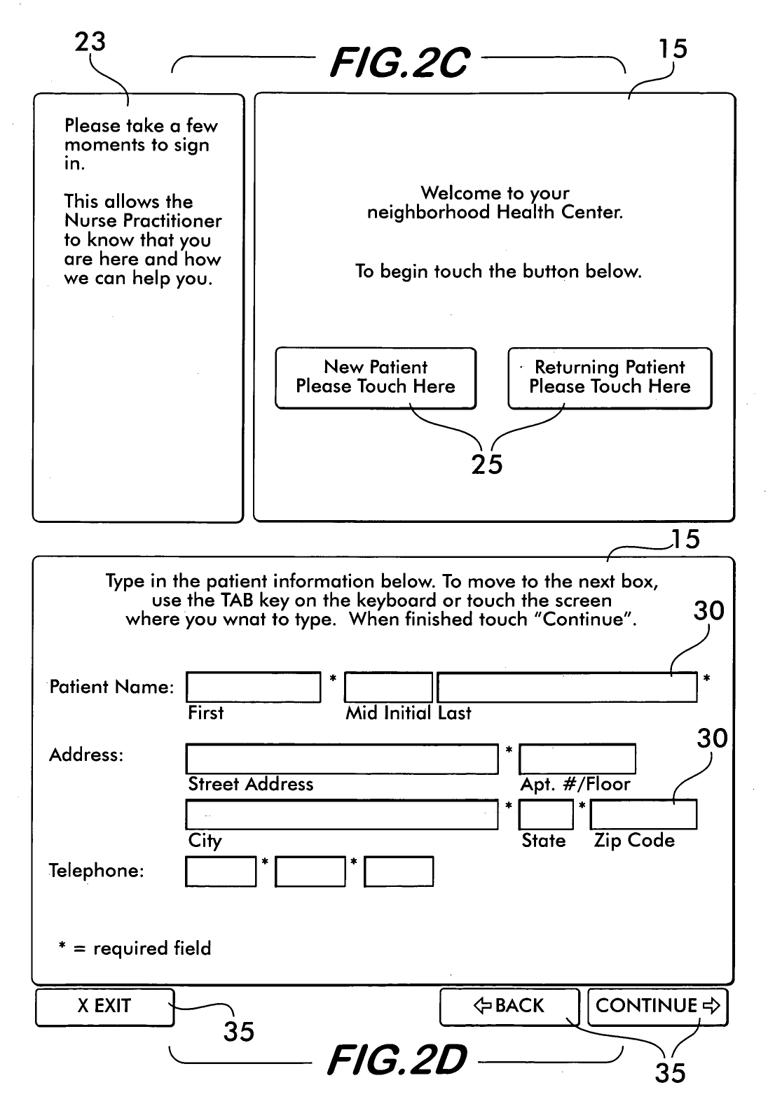

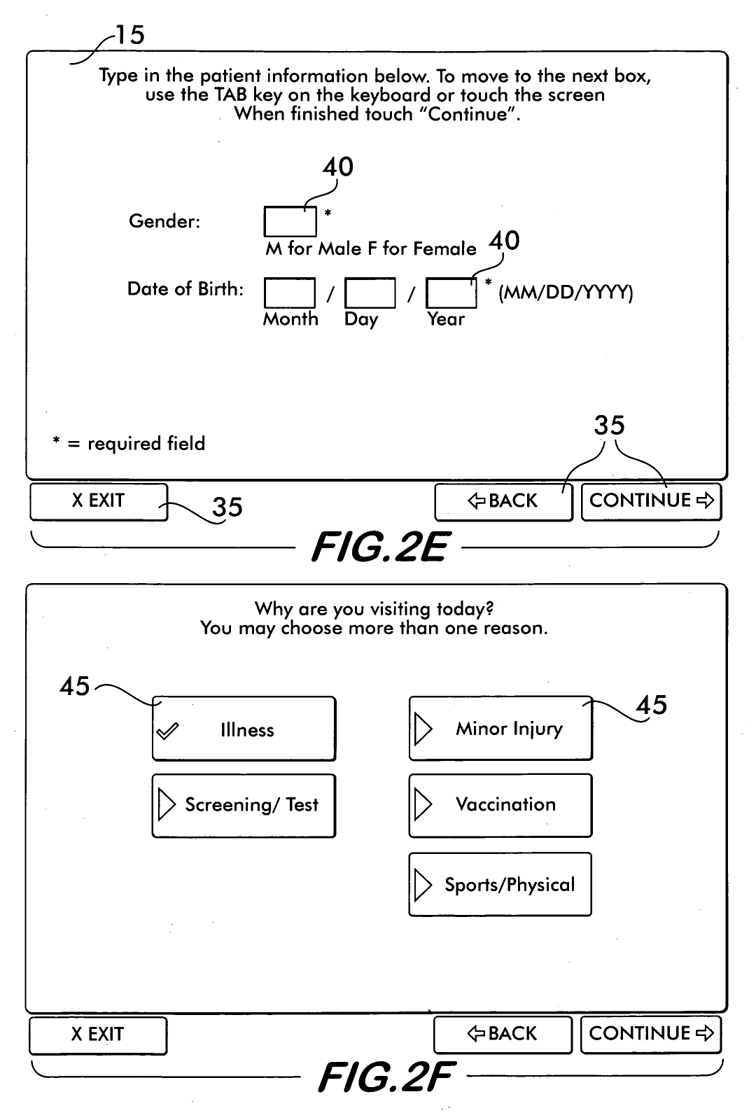

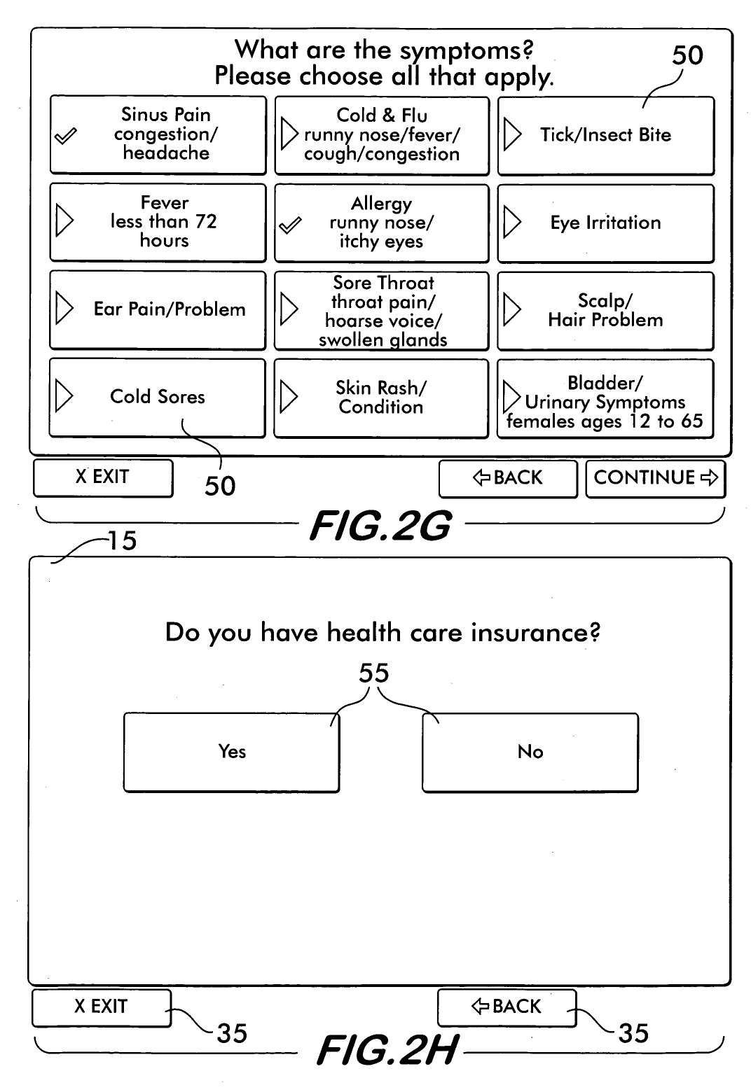

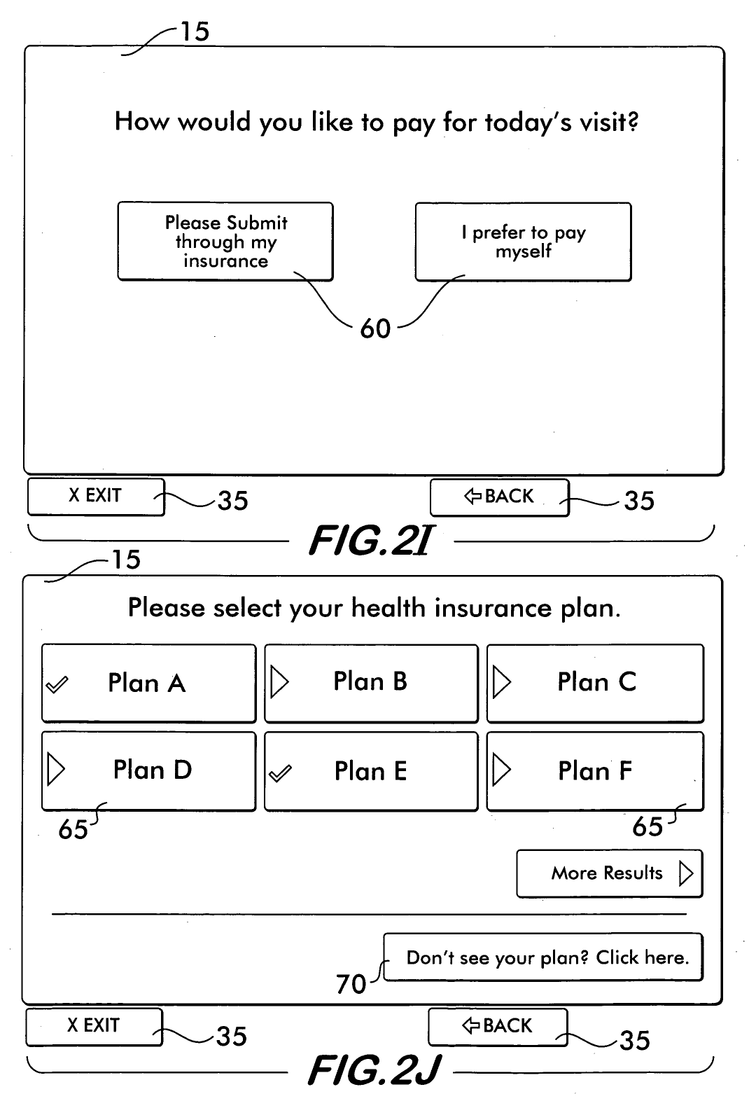

Referring now to FIGS. 2A-2J, there is shown a series of photographs and screen shots illustrating the system and method of the present invention according to a preferred embodiment. As previously described, a kiosk 13 can be provided within the patient care system 10 in order to permit the patient 12 to register with the patient care system 10 thereby signaling the system 10 that an encounter is being initiated. The kiosk 13 can be a permanent structure for maintaining access to the patient care system 10 from a selected location for a long period or time. However, in one preferred embodiment of the invention the kiosk 13 can be a temporary structure or a portable structure, for permitting the kiosk 13 to be conveniently assembled at a location for a relatively shorter period, disassembled, removed, transported, and reassembled at another location. For example, the kiosk 13 can be a pop-up kiosk for permitting convenient assembly, disassembly and transporting thereof.

Furthermore, permanent kiosks 13 as well as temporary or portable kiosks 13 in accordance with the present invention can be provided at any location that is suitable for an encounter between a patient 12 and a clinician 18, provided the location permits access to the internet 16, in order to permit communication between the kiosk 13 and the medical records system 17. In one preferred embodiment of the invention a kiosk 13 can be located within or in the vicinity of a pharmacy in order to save the time required by the patient 12 to travel to a pharmacy to fill a prescription for medication or equipment after an encounter.

As shown in FIG. 2A, the kiosk 13 can include a display device 15 for permitting the patient 12 to interact conveniently with the electronic medical records system 17. As previously described, the kiosk system including the kiosk 13 is fully integrated with the medical records system 17, preferably by way of the internet 16. Additionally, in a preferred embodiment of the invention, the kiosk 13 can include a display of instructions 20, or a plurality of displays of instructions 20, in order to assist the patient 12 in using the patient care system 10.

The display device 15 provided within a kiosk 13 can display a plurality of buttons 25 as shown in FIG. 2B. The buttons 25 can permit the patient 12 to initiate an encounter with the patient care system 10 and to indicate to the medical records system 17 whether he/she is a new patient or a returning patient. Accordingly, the medical records system 17 can retrieve previously stored information for the patient 12 if a determination is made that such information exists. Additionally, the display device 15 can include a viewing pane 23 for providing additional instructions to assist the patient 12 in interacting with the health care system 10, as shown more clearly in FIG. 2C.

When the patient 12 indicates that he/she is a new patient or a returning patient by way of the buttons 25, the display device 15 can provide a display of a plurality of data fields 30 as shown in FIG. 2D. The data fields 30 can be used by the patient 12 to enter name, address and telephone number information into the medical records system 17. The queries put to a patient 12 by the medical records system 17, and the information provided by the patient 12, can include any queries or information that may facilitate the encounter between the patient 12 and the patient care system 10. Additionally, the buttons 35 can be provided on the display device 15 in order to permit the patient 12 to return to a previously displayed screen, move to the next display screen, or exit from the electronic patient care system 10.

In the next screen displayed on the display device 15, additional identifying information can be entered by the patient 12 by way of the data fields 40 as shown in FIG. 2E. The information entered into the data fields 40 by the patient 12 can be gender information, social security information, or any other kind of information known to those skilled in the art. When the information of the data fields 30, 40, or when any other identifying information is received, the medical records system 17 can request that the patient 12 enter information regarding the nature of the current encounter. In order to enter this information the patient 12 can use the buttons 45 as shown in FIG. 2F. For example, using the buttons 45 on the display device 15 the patient 12 can indicate that the current encounter is being initiated because of an illness, a minor injury, a screening/test, a vaccination or a sports physical. Additionally, further buttons 45 may be provided for any other reason which may be useful for determining how the clinician 18 should treat the patient 12 during the encounter.

As shown in FIG. 2G, a further screen may be provided on the display device 15 wherein the buttons 50 can be used by the patient 12 to indicate the symptoms of the patient 12 to the patient care system 10. For example, using the buttons 50 the patient 12 can indicate sinus pain, cold and flu, a tick/insect bite, a fever (less than 72 hours), an allergy, an eye irritation, an ear pain/problem, a sore throat, a scalp/hair problem, cold sores, a skin rash/condition, bladder/urinary symptoms, or any other symptoms

Furthermore, any type of symptoms can be indicated to the patient care system 10. For example, the indicated symptoms can be very specific information required by medical specialists, such as cardiologists. Furthermore, the symptoms can be indicated in any manner known to those skilled in the art. For example, the symptoms can be indicated by selecting or creating images of any type that may be determined to be useful for an encounter within the patient care system 10. Images can be displayed on the display device 15 to permit, for example, the patient 12 to point to, to circle or to draw an area of the body where pain or other concerns are localized.

It will be understood by those of ordinary skill in the art that the clinician 18 must operate strictly according to guidelines that are promulgated for patient care within the patient care system 10. The guidelines are adapted to strictly comply with all applicable laws, codes and regulations. For example, the clinician 18 may be a physician, a nurse practitioner or any other health care worker. The governing state law where the kiosk 13 is located may set forth which conditions a clinician 18 is permitted to treat without collaborating with a physician. The guidelines may list any conditions that must be treated only if a physician collaborates during an encounter. Furthermore, the guidelines can set forth which conditions can be treated at the time of the encounter by the clinician 18 but must be submitted for later review by a collaborating physician. Conditions which must be immediately referred to an emergency room can also be indicated.

As an example, a nurse clinician 18 may be required to refer a patient 12 having a fever longer than seventy-two hours to an emergency room rather than trying to treat him/her. In a preferred embodiment, the clinician 18 can be alerted immediately when a patient 12 indicates a condition which should be handled by the emergency room. Furthermore, the clinician 18 may be provided with predetermined protocols for all conditions treated during an encounter within the patient care system 10 and required to strictly comply with them.

After indicating his/her symptoms to the health care system 10 using the buttons 50, the patient 12 can indicate whether he/she has health care insurance using the buttons 55 in a further screen shown on the display device 15. The buttons 50 are shown in FIG. 2H. Additionally, the patient 12 can indicate whether payment for the current encounter with the patient care system 10 should be submitted through an insurance company, or paid by the patient 12 using the buttons 60 on the display device 15 as shown in FIG. 2I. Any information entered into the health care system 10 by buttons or any other method described herein can also be collected using a of a card such as a credit card or an insurance card, an RFID device, an optical device or any other information device.

Furthermore, in one preferred embodiment of the invention, the patient 12 can be prompted by the patient care system 10 to indicate which health insurance plan is to be used for payment using buttons 65 or any other device, as shown in FIG. 2J. As also shown in FIG. 2J, additional health insurance plans or additional instructions for the patient 12 can be selected using the button 70 on the display device 15 if necessary.

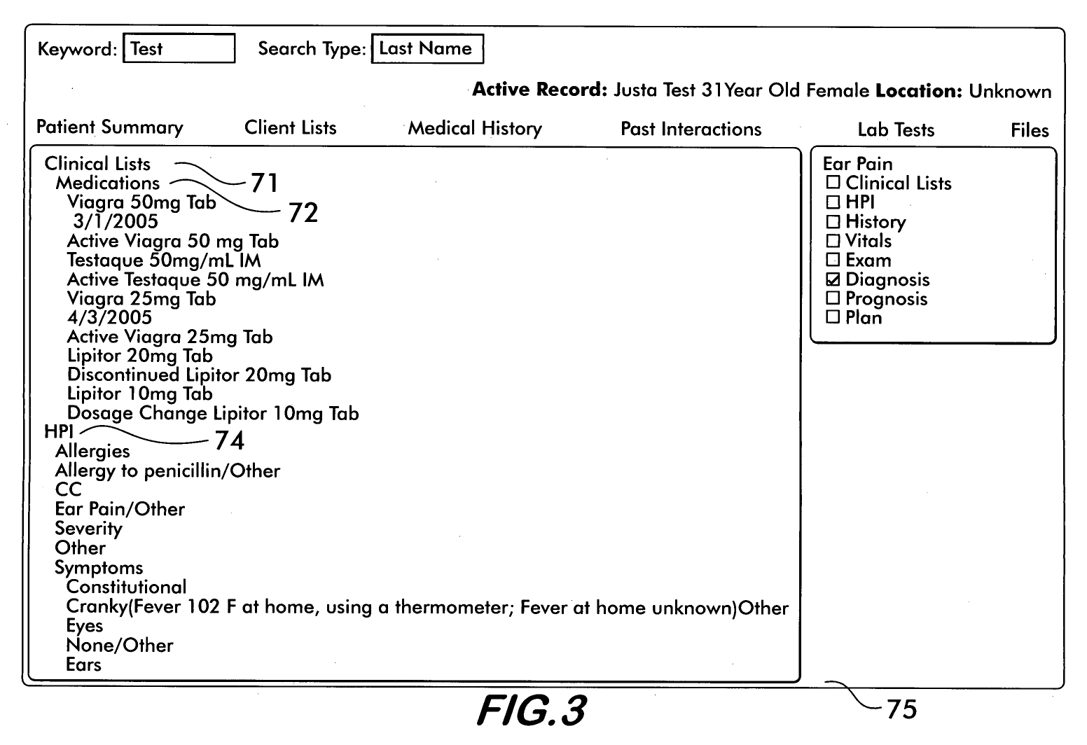

Referring now to FIG. 3, there is shown a screen shot of a graphical user interface 75 suitable for collecting or entering any information and for displaying any information. The information displayed can include any guidelines or protocols that may be relevant to the encounter for the clinician 18 or the patient 12. The display of FIG. 3 shows one example of the kind of data from the electronic medical records system 17 which can be available to the clinician 18 during an encounter. However, the graphical user interface 75 can display any kind of information for the clinician 18 that may facilitate an encounter between a patient 12 and the patient care system 10. It will be understood that such a display of data from the electronic medical records system 17 can eliminate the need for pulling hard copies of the patient's records from shelves.

As also shown in FIG. 3 the graphical user interface 75 can set forth a plurality of clinical lists 71 for the clinician 18. For example, a history of the medication prescribed for the patient 12 can be shown in a medications list 72. Additionally, a History of Present Illness (HPI) list 74 can be provided to indicate. For example, any allergies that the patient 12 may have, along with the severity and the patient's symptoms can be displayed. Any other problems that the clinician 18 should be aware of can also be shown on the graphical user interface 75 in this manner. It will be understood that current information entered by the patient into the patient care system 10, and received by the medical records system 17, can be made available to the clinician 18 at the location of the kiosk system instantaneously, along with the information on the patient 12 that was previously stored in the medical records system 17. Furthermore, any information entered into the medical records system 17 by the clinician during an encounter can also be displayed instantaneously on the graphical user interface 75.

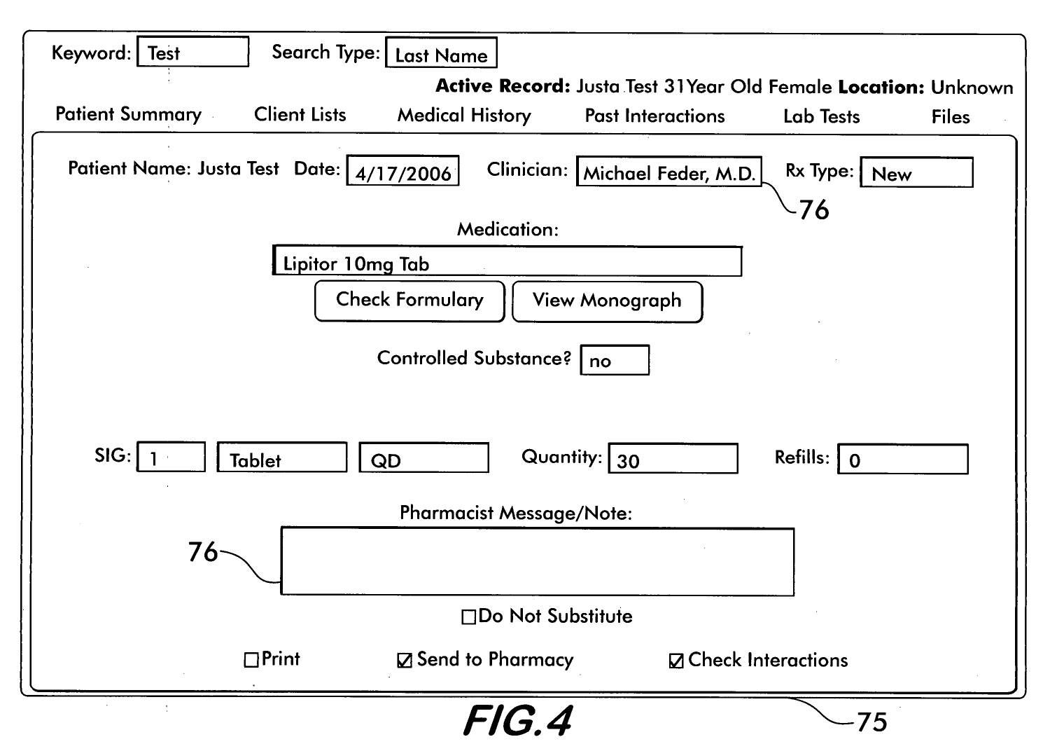

Referring now to FIG. 4, a prescription form can be provided to the clinician 18 on the graphical user interface 75 for prescribing any required medicines or services for the patient 12 in accordance with the encounter between the patient 12 and the clinician 18 within the patient care system 10. For example, the name of a clinician 18, the name of a medication and the prescription instructions can be entered in data fields 76 displayed on the graphical use interface 75 as shown.

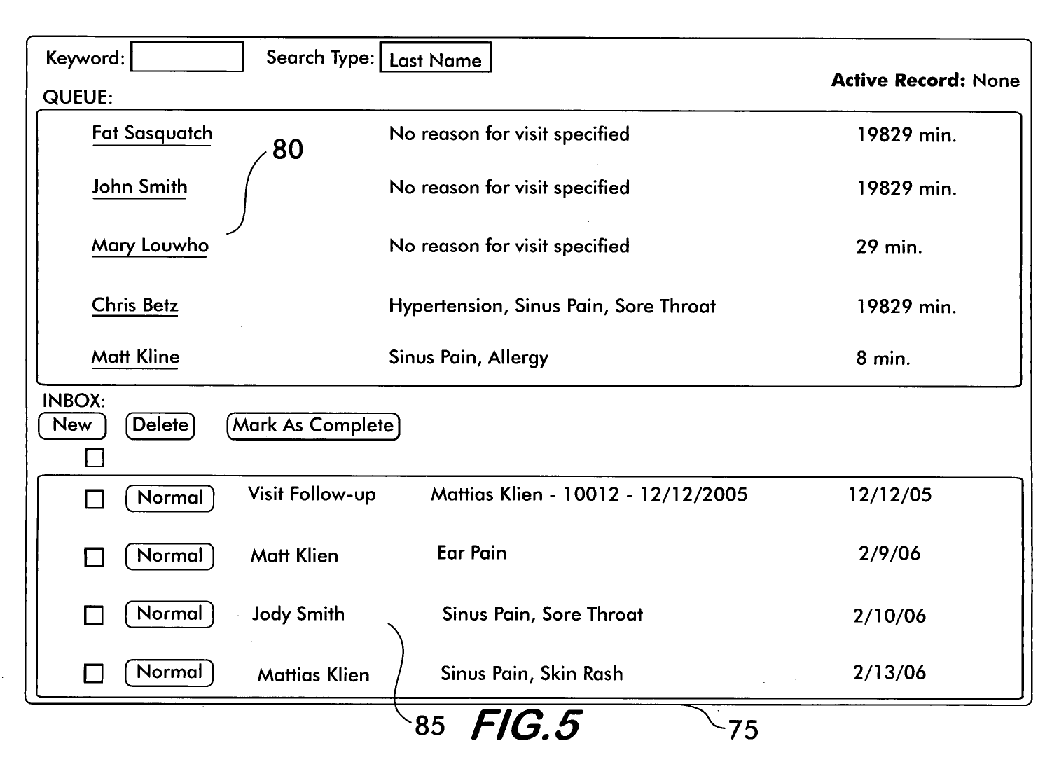

As shown in FIG. 5, additional information can be displayed on the graphics user interface 75. The additional information can include a list of the patients 12 in the queue of the clinician 18 who are waiting to see the clinician 18. This information is available to the corporate headquarters 21 at all times for all kiosks 13 that are active in the patient care system 10.

User inbox 85 can contain any tasks, reminders, or instructions that the user of the system may find useful. These items can be created by the patient 12 of, the system 10 (based on workflow rules or any other source of rules), or another user. The items can be provided with a priority level (i.e. high, medium, low, etc.) and can be tied to various workflow rules or any other source of rules. For example, if new medical results are received the results can be analyzed by the system 10 and a task can be created by the system 10 to that indicate when new abnormal lab results have arrived. In another example, after a patient 12 has been seen, the system 10 can create a visit follow-up task to indicate that a call should be made to the patient 12 back to determine how he/she are doing.

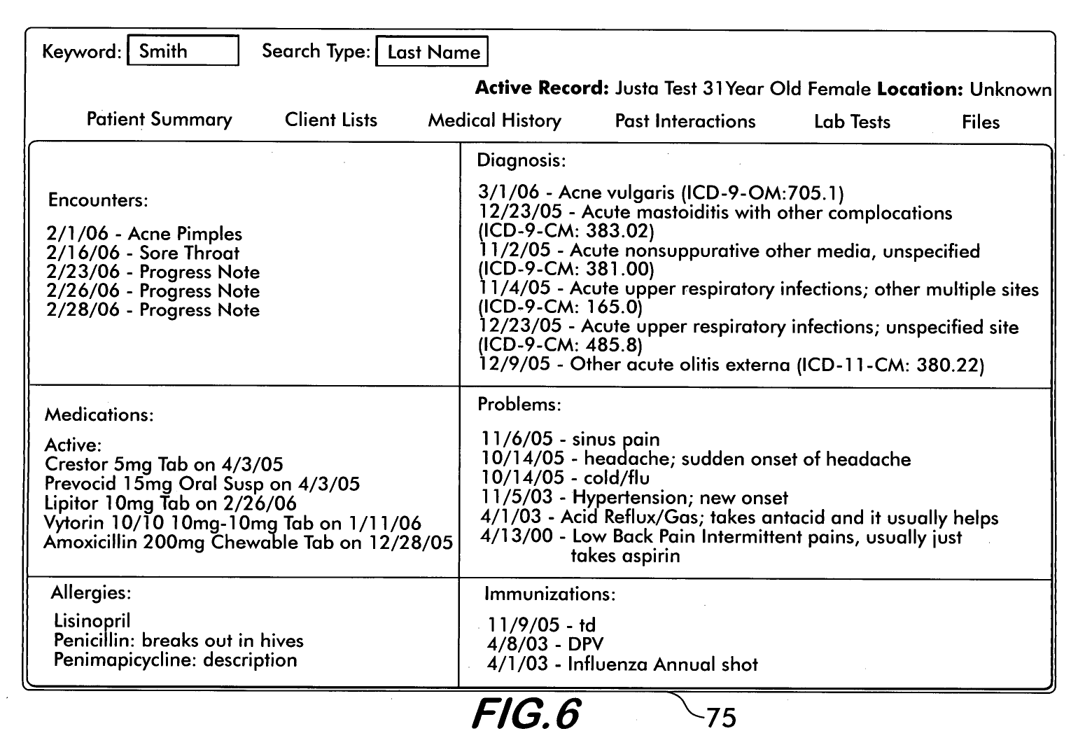

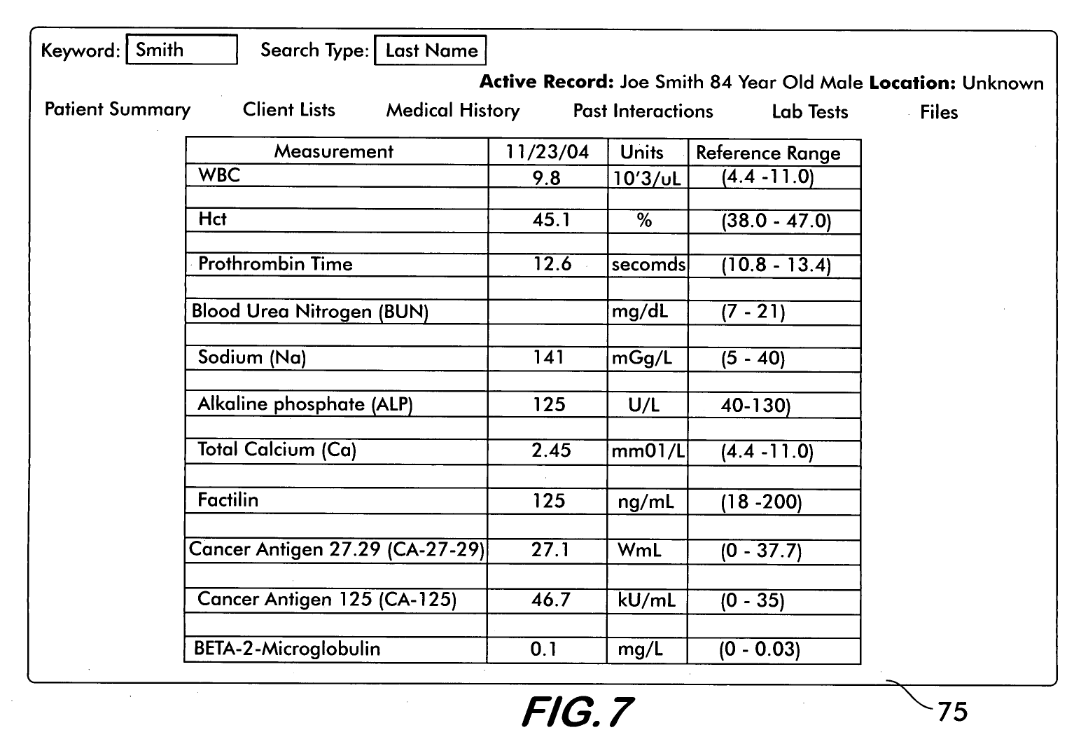

Additionally, the clinician 18 can be provided with a screen on the graphical interface user 75 showing a face sheet (or encounter history) of a patient 12 as shown in FIG. 6. The face sheet can be a high level view of the history of the patients 12. Each encounter between the patient 12 and the patient care system can be displayed, along with its corresponding symptoms. Likewise, the problems presented by the patient 12 in the previous encounters with the patient care system 10 and the medications provided, can be displayed on the graphical user interface 75. Furthermore, information on allergies, immunizations and any other information believed to be useful by those skilled in the art for diagnosing and treating the patient 12 by the clinician 18 can be displayed on the graphical user interface 75. Likewise, the results of clinical tests performed on the patient 12 can be displayed on the graphical user interface 75 as shown in FIG. 7.

While the invention has been described in detail and with reference to specific examples thereof, it will be apparent to one skilled in the art that various changes and modifications can be made therein without departing from the spirit and scope thereof.

What is claimed is:

1. A patient care system for providing health care to a patient, comprising:
a clinician for providing said health care to said patient;
an input device for signaling by said patient that said patient is initiating a current health care encounter to provide a patient-initiated encounter signal; and
current patient information provided by said patient and received by said patient care system in response to said patient-initiated encounter signal.

2. The patient care system for providing health care to a patient of claim 1, further comprising an interface device in communication with said input device for providing a clinician-notification signal in response to said patient-initiated encounter signal to permit said clinician to determine that said patient has initiated said current health care encounter.

3. The patient care system for providing health care to a patient of claim 1, further comprising an electronic medical records system in communication with said input device for receiving said current patient information from said patient.

4. The patient care system for providing health care to a patient of claim 3, further comprising a plurality of input devices in communication with said electronic medical records system for receiving said current patient information from a plurality of differing patients.

5. The patient care system for providing health care to a patient of claim 4, wherein said differing patients of said plurality of differing patients are disposed at a plurality of differing locations.

6. The patient care system for providing health care to a patient of claim 3, further comprising corporate headquarters for receiving said current patient information and managing said patient care system in accordance with said current patient information.

7. The patient care system for providing health care to a patient of claim 6, further comprising previous patient information stored in said electronic medical records system received by said corporate headquarters from said electronic medical records system for managing said patient care system in accordance with said previous patient information.

8. The patient care system for providing health care to a patient of claim 7, wherein said corporate headquarters manages said patient care system in accordance with both said current patient information and said previous patient information.

9. The patient care system for providing health care to a patient of claim 3, wherein said electronic medical records system is in communication with said input device by way of an internet.

10. The patient care system for providing health care to a patient of claim 9, wherein said electronic medical records system is in communication with said clinician by way of said internet.

11. The patient care system for providing health care to a patient of claim 3, further comprising current encounter information received by said clinician from said electronic medical records system in response to said patient-initiated encounter signal.

12. The patient care system for providing health care to a patient of claim 3, further comprising previous encounter information in accordance with a previous encounter said patient received by said clinician from said electronic medical records system in response to said patient-initiated encounter signal.

13. The patient care system for providing health care to a patient of claim 12, further comprising current encounter information received by said clinician from said electronic medical records system in response to said patient-initiated encounter signal.

14. The patient care system for providing health care to a patient of claim 1, further comprising a kiosk for housing said input device.

15. The patient care system for providing health care to a patient of claim 14, further comprising a permanent kiosk.

16. The patient care system for providing health care to a patient of claim 14, further comprising a temporary kiosk.

17. The patient care system for providing health care to a patient of claim 16, further comprising a pop up kiosk.

18. The patient care system for providing health care to a patient of claim 1, wherein said current patient information comprises patient identification information.

19. The patient care system for providing health care to a patient of claim 1, wherein said current patient information comprises patient symptom information.

20. The patient care system for providing health care to a patient of claim 1, wherein said current patient information comprises patient insurance information.
